(19) United States
(12) Patent Application Publication
(10) Pub. No.: US 2020/0002479 A1
(43) Pub. Date: Jan. 2, 2020

(54) CLICK NUCLEIC ACID POLYMERS AND METHODS OF MAKING

(71) Applicant: THE REGENTS OF THE UNIVERSITY OF COLORADO, A BODY CORPORATE, Denver, CO (US)

(72) Inventors: Christopher N. BOWMAN, Boulder, CO (US); Xun HAN, Boulder, CO (US); Benjamin D. FAIRBANKS, Boulder, CO (US)

(73) Assignee: THE REGENTS OF THE UNIVERSITY OF COLORADO, A BODY CORPORATE, Denver, CO (US)

(21) Appl. No.: 16/454,594

(22) Filed: Jun. 27, 2019

Related U.S. Application Data
(60) Provisional application No. 62/692,563, filed on Jun. 29, 2018.

(51) Int. Cl. C08G 75/045; A61K 47/69; A61K 47/02; C08G 85/00

(52) U.S. Cl. CPC C08G 75/045; A61K 47/6929; B82Y 5/00; C08G 85/002; A61K 47/02

(57) ABSTRACT

Disclosed herein are Click Nucleic Acid Polymers (CNA-polymers) that comprise one or more units which can anneal to complementary units in a manner which affords hybridization of the disclosed CNA's to synthetic or naturally occurring polymers. Also disclosed are nanoparticle/CNA conjugate. Further disclosed are methods for preparing the disclosed polymers and nanoparticle/polymer conjugates.

### CLAIM TO PRIORITY

This application claims priority to U.S. Provisional Application Ser. No. 62/692,563, filed Jun. 29, 2018, the entirety of which is included herein by reference.

### GOVERNMENT SUPPORT

This invention was made with government support under grant number DMR1420736 awarded by The National Science Foundation. The government has certain rights in the invention.

### FIELD OF THE DISCLOSURE

Disclosed herein are Click Nucleic Acid Polymers (CNA-polymers) that comprise one or more units which can anneal to complementary units in a manner which affords hybridization of the disclosed CNA's to synthetic or naturally occurring polymers. Also disclosed are nanoparticle/CNA conjugate. Further disclosed are methods for preparing the disclosed polymers and nanoparticle/polymer conjugates.

### BRIEF DESCRIPTION OF THE FIGURES

FIG. 1 is a ¹H NMR of a disclosed polyT (polythymine)CNA.

FIG. 2 is an enlarged ¹H NMR of the polyT (polythymine)CNA of FIG. 1 wherein the region from 4.5 to 7.5 ppm is amplified to show the terminal vinyl protons.

FIG. 3A depicts the photopolymerization efficiency of the polymerization at various reaction times as determined by size exclusion chromatograpy (SEC)

FIG. 3B depicts the photopolymerization efficiency of the polymerization at various reaction times as determined by Fourier Transform Infrared (FTIR) spectroscopy.

FIG. 4 depicts the interaction between polyT CNA polymers and ssDNA as evaluated by gel electrophoresis.

FIG. 5 depicts the intensity of the unbound DNA band from FIG. 4 as plotted against the concentration of loaded polyT CNA wherein these data were fit to a sigmoidal curve in GraphPad Prism.

FIG. 6 depicts the specificity of the disclosed polyT CNA polymer units to anneal with DNA, and therefore the polymer to hybridize. These data were determined by mixing polyT CNA with complementary and non-complementary ssDNA.

FIG. 7 depicts the hybridization of a disclosed nanoparticle/polyT CNA polymer conjugate to both strands of a section of DNA having repeating polyA (adenine) units.

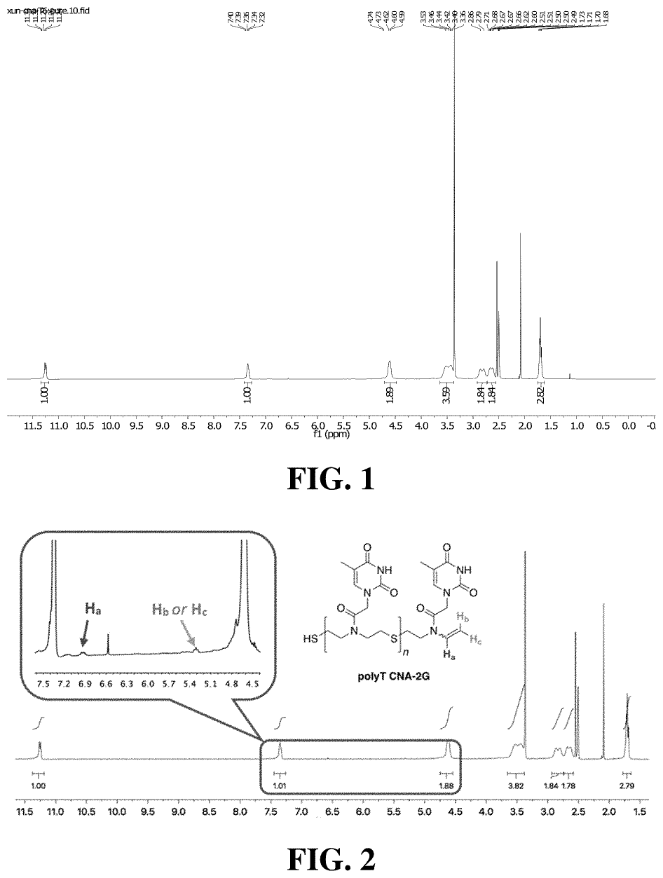

### DETAILED DESCRIPTION

In this specification and in the Claim that follow, reference will be made to a number of terms, which shall be defined to have the following meanings:

All percentages, ratios and proportions herein are by weight, unless otherwise specified. All temperatures are in degrees Celsius (o C) unless otherwise specified.

Throughout the description and Claim of this specification the word “comprise” and other forms of the word, such as “comprising” and “comprises,” means including but not limited to, and is not intended to exclude, for example, other additives, components, integers, or steps.

As used in the description and the appended Claim, the singular forms “a,” “an,” and “the” include plural referents unless the context clearly dictates otherwise.

The term “residue” or “backbone unit” means an element of an oligomer or polymer that derives from a monomer. For example, below is a residue that results in a backbone unit and the monomer from which the residue or backbone unit is derived.

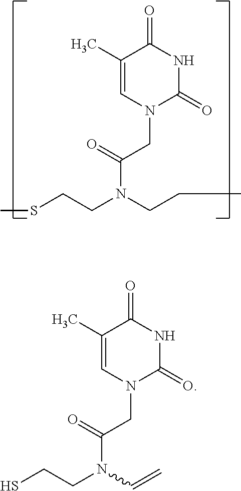

Disclosed herein are monomers which can be assembled into oligomers having two or more residues. Subsequently the oligomers can be polymerized to afford CNA-polymers having repeating sequences of the desired oligomers.

### Click Nucleic Acid Polymers

Disclosed herein are monomers which can be used to prepare the disclosed Click Nucleic Acid Polymers (CNA's) and the nanoparticle CNA's.

One aspect of the disclosed polymers comprise backbone units or residues comprising a nucleobase. The disclosed nucleobase comprising monomers can be prepared by the procedure outlined in Scheme I and described in Example 1 below.

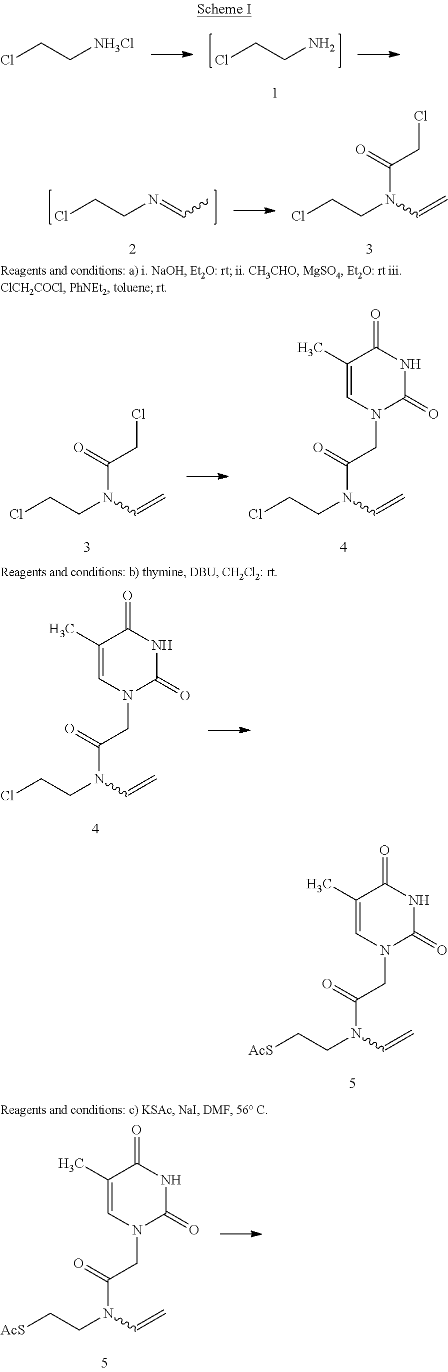

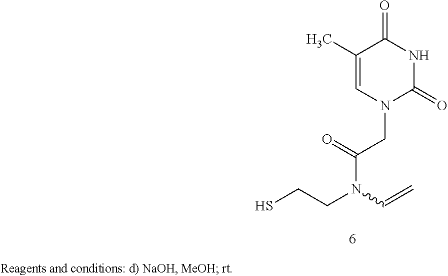 [SMILES: C=CN(CCS)C(=O)Cn1cc(C)c(=O)[nH]c1=O]

### Example 1

### N-(2-mercaptoethyl)-2-(5-methyl-2,4-dioxo-3,4-dihydropyrimidin-1(2H)-yl)-N-vinylacetamide (6)

Preparation of 2-chloro-N-(2-chloroethyl)-N-vinylacetamide, 3: To a solution of 2-chloroethylamine hydrochloride (35 g, 0.30 mol) in water (50 mL) was added NaOH (18 g, 0.40 mol) in portions. The resulting solution was extracted with diethyl ether (3×100 mL). The combined organic layers were dried over MgSO₄, then added to a 1 L round-bottom flask filled with acetaldehyde (26 g, 0.59 mol), 200 mL diethyl ether, and MgSO₄ (40 g, 0.33 mol) at room temperature. The resulting mixture was stirred at room temperature for 30 min, then filtered. The filtrate was concentrated in vacuum and re-dissolved in toluene (300 mL). To fresh imine toluene solution were added chloroacetyl chloride (20 mL, 0.25 mol) and N,N-diethylaniline (40 mL, 0.25 mol). The resulting brown mixture was stirred at room temperature for 1 hour before quenched with water. The organic layer was separated and dried over MgSO₄, filtered, and concentrated. The residue was purified by flash column chromatography (hexanes:EtOAc=20:1 to 4:1) in silica gel to afford desired product (30 g, 66% yield, mixture of two diastereomers, d.r.=4.0:1) as a pale yellow oil. ¹H NMR (400 MHz, CDCl₃): δ 7.33 (dd, 0.2H, J=6.0, 14.0 Hz), 6.74 (dd, 0.8H, J=6.0, 14.0 Hz), 4.74 (dd, 0.8H, J=2.0, 14.0 Hz), 4.61-4.58 (m, 1.2H), 4.30 (s, 0.4H), 4.20 (s, 1.6H), 3.98 (t, 2H, J=6.0 Hz), 3.74 (t, 0.4H, J=8.0 Hz), 3.62 (t, 0.4H, J=6.0 Hz); ¹³C NMR (100 MHz, CDCl₃): δ 165.5, 132.2, 130.6, 97.7, 95.9, 45.9, 44.6, 41.7, 41.3, 40.0, 39.1; HRMS (ESI) m/z calculated for C₆H₉Cl₂NO [M+H]⁺ 182.0139, found 182.0142.

Preparation of N-(2-chloroethyl)-2-(5-methyl-2,4-dioxo-3,4-dihydropyrimidin-1(2H)-yl)-N-vinylacetamide, 4: To a suspension of thymine (6.9 g, 55 mmol) and compound 3 (10 g, 55 mmol) in CH₂Cl₂ (500 mL) was added 1,8-diazabicyclo(5.4.0)undec-7-ene (9.5 mL, 60 mmol) dropwise. The resulting mixture was stirred at room temperature for 5 hours, then charged silica gel. The solvent was removed under vacuum. A dry-pack flash column chromatography (CH₂Cl₂:MeOH=100:0 to 10:1) was set up to produce desired pure nucleotide 4 (6.8 g, 46%, mixture of two diastereomers, d.r.=2.2:1) as a white powder. ¹H NMR (400 MHz, DMSO-d₆): δ 11.34 (s, 1H), 7.41 (s, 1H), 7.19 (dd, 0.31H, J=8.0, 16.0 Hz), 6.96 (dd, 0.69H, J=8.0, 16.0 Hz), 4.82-4.73 (m, 3H), 4.52 (d, 1H, J=12.0 Hz), 3.99 (t, 0.62H, J=6.0 Hz), 3.95 (t, 1.38H, J=6.0 Hz), 3.84 (t, 0.62H, J=6.0 Hz), 3.66 (t, 0.62H, J=6.0 Hz), 1.75 (s, 3H); ¹³C NMR (100 MHz, DMSO-d₆): δ 166.4, 164.4, 151.0, 142.0, 131.6, 130.1, 108.3, 96.3, 95.5, 48.7, 44.1, 43.1, 40.3, 39.8, 12.0; HRMS (ESI) m/z calculated for C₁₁H₁₄ClN₃O₃[M+Li]⁺278.0884, found 278.0882.

Preparation of S-(2-(2-(5-methyl-2,4-dioxo-3,4-dihydropyrimidin-1 (2H)-yl)-N-vinylacetamido)ethyl) ethanethioate, 5: To a solution of compound 4 (1.1 g, 4.0 mmol) in DMF (5 mL) were added potassium thioacetate (1.4 g, 12 mmol) and NaI (30 mg, 0.20 mmol) successively. The resulting mixture was stirred at 55° C. overnight, then the solvent was removed under vacuum. The residue was diluted with CH₂Cl₂ (20 mL), then charged with silica gel. The mixture was dried under vacuum. A dry-pack flash column chromatography (CH₂Cl₂:MeOH=100:0 to 10:1) was set up to afford acetyl protected monomer 5, which was further purified through recrystallization from EtOH/water (1/1) to yield pure desired product (914 mg, 63% yield, mixture of two diastereomers, d.r.=1.7:1) as a little brown powder. ¹H NMR (400 MHz, DMSO-d₆): δ 11.34 (s, 1H), 7.43 (s, 0.37H), 7.40 (s, 0.63H), 7.19 (dd, 0.37H, J=8.0, 16.0 Hz), 6.95 (dd, 0.63H, J=8.0, 16.0 Hz), 4.93-4.88 (m, 1H), 4.81 (s, 0.74H), 4.74 (s, 1.26H), 4.55-4.50 (m, 1H), 3.73-3.65 (m, 2H), 3.11 (t, 0.74H, J=8.0 Hz), 2.93 (t, 1.26H, J=8.0 Hz), 2.39 (s, 1.11H), 2.34 (s, 1.89H), 1.75 (s, 3H); ¹³C NMR (100 MHz, DMSO-d₆): δ 195.8, 195.3, 166.0, 164.4, 151.0, 142.0, 142.0, 131.6, 130.3, 108.3, 95.5, 95.1, 54.9, 48.6, 42.4, 41.0, 30.5, 25.3, 24.9; HRMS (ESI) m/z calculated for C₁₃H₁₇N₃O₄S [M+Li]⁺318.1100, found 318.1104.

Preparation of N-(2-mercaptoethyl)-2-(5-methyl-2,4-dioxo-3,4-dihydropyrimidin-1(2H)-yl)-N-vinylacetamide, 6: To a suspension of compound 5 (330.5 mg, 1.06 mmol) in MeOH (3 mL) was added 4.5 M NaOH aqueous solution (0.5 mL, 2.25 mmol) in one portion. The resulting mixture was stirred at room temperature for 5 min, at which it turned to a brown clear solution. Then 1 M HCl aqueous solution was slowly added to neutralize the reaction solution (pH z 7). Meanwhile, precipitates came out and was collected by filtration, then washed with acetone twice to afford pure monomer (246.5 mg, 87% yield, mixture of two diastereomers, d.r.=1.7:1) as a little brown powder. ¹H NMR (400 MHz, DMSO-d₆): δ 11.34 (s, 1H), 7.42 (s, 0.37H), 7.41 (s, 0.63H), 7.19 (dd, 0.37H, J=8.0, 16.0 Hz), 6.92 (dd, 0.63H, J=8.0, 16.0 Hz), 4.78-4.70 (m, 3H), 4.51-4.47 (m, 1H), 3.72 (t, 2H, J=8.0 Hz), 2.76-2.68 (m, 0.74H), 2.61-2.52 (m, 1.26H), 1.75 (s, 3H); ¹³C NMR (100 MHz, DMSO-d₆): δ 165.9, 164.4, 151.0, 142.0, 131.6, 130.3, 108.2, 95.6, 95.0, 48.8, 48.6, 45.9, 44.8, 20.7, 20.2, 11.9; HRMS (ESI) m/z calculated for Cl₁H₁₅N₃O₃S [M+H]⁺ 270.0912, found 270.0914.

Other disclosed monomers can be prepared according to Scheme I by substitution of the appropriate nucleobase for thymine in step (b) of the sequence. For example, 2-(4-amino-2-oxo-3,4-dihydropyrimidin-1 (2H)-yl)-N-(2-mercaptoethyl)-N-vinvlacetamide having the formula:

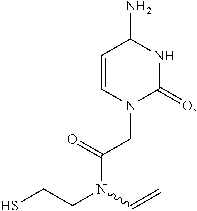 [SMILES: C=CN(CCS)C(=O)CN1C=CC(N)NC1=O]

can be prepared by substituting cytosine for thymine in the procedure of Example 1.

2-(2,4-Dioxo-3,4-dihydropyrimidin-1 (2H)-yl)-N-(2-mercaptoethyl)-N-vinylacetamide having the formula:

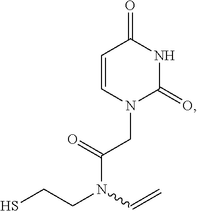 [SMILES: C=CN(CCS)C(=O)Cn1ccc(=O)[nH]c1=O]

can be prepared by substituting cytosine for uracil in the procedure of Example 1.

2-(6-Amino-9H-purin-9-yl)-N-(2-mercaptoethyl)-N-vinylacetamide having the formula:

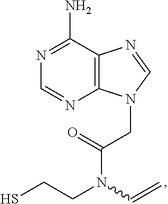 [SMILES: C=CN(CCS)C(=O)Cn1cnc2c(N)ncnc21]

can be prepared by substituting adenine for thymine in the procedure of Example 1.

2-(2-Amino-6-oxo-1H-purin-9(6H)-yl)-N-(2-mercaptoethyl)-N-vinylacetamide having the formula:

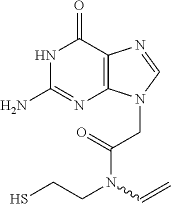 [SMILES: C=CN(CCS)C(=O)Cn1cnc2c(=O)[nH]c(N)nc21]

can be prepared by substituting adenine for guanine in the procedure of Example 1.

In one aspect the disclosed polymers have the formula:

[X₁]—[X₂]ₘ—[X₃]

wherein X₁ has the formula:

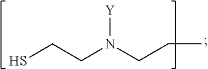

X₂ has the formula:

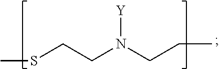

X₃ has the formula:

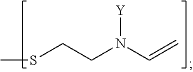

Y has the formula:

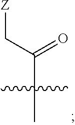

each Z is the same or a different moiety capable of annealing to a complementary moiety; and the index m is an integer from 0 to 20. The index m can have any value from 0 to 20, for example, 0, 1, 2, 3, 4, 5, 6, 7, 8, 9, 10, 11, 12, 13, 14, 15, 16, 17, 18, 19, and 20.

X₁ units are referred to herein as “thiol terminal units” or alternatively “S terminal units.” X₃ units are referred to herein as “vinyl terminal units” or alternatively “terminal units having a terminal double bond.” X₃ units can be referred to herein, for example, as “block units wherein Z is thymine,” or “poly-T block units,” or “poly-TC block units,” etc.

In one embodiment, Z is a nucleobase. Non-limiting examples of suitable nucleobases includes cytosine, guanine, adenine, thymine, uracil, 5-fluorouracil, 5-bromouracil, 5-chlorouracil, 5-iodouracil, hypoxanthine, xanthine, acetylcytosine, 5-(carboxyhydroxyl-methyl)uracil, 5-carboxymethylaminomethyl-2-thiouridine, 5-carboxymethylamino-methyluracil, dihydrouracil, β-D-galactosyl-queosine, inosine, 1-methylguanine, 1-methyl-inosine, 2,2-dimethylguanine, 2-methyladenine, 2-methylguanine, 3-methyl-cytosine, 5-methyl-cytosine, N⁶-adenine, 7-methylguanine, 5-methylaminomethyluracil, methoxyamino-methyl-2-thiouracil, beta-D-mannosylqueosine, 5′-methoxycarboxy-methyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine.

In one iteration of this embodiment, each Z is independently chosen from adenine, guanine, thymine, uracil and cytosine. The following are non-limiting example of CNA's wherein each Z is the same nucleobase:

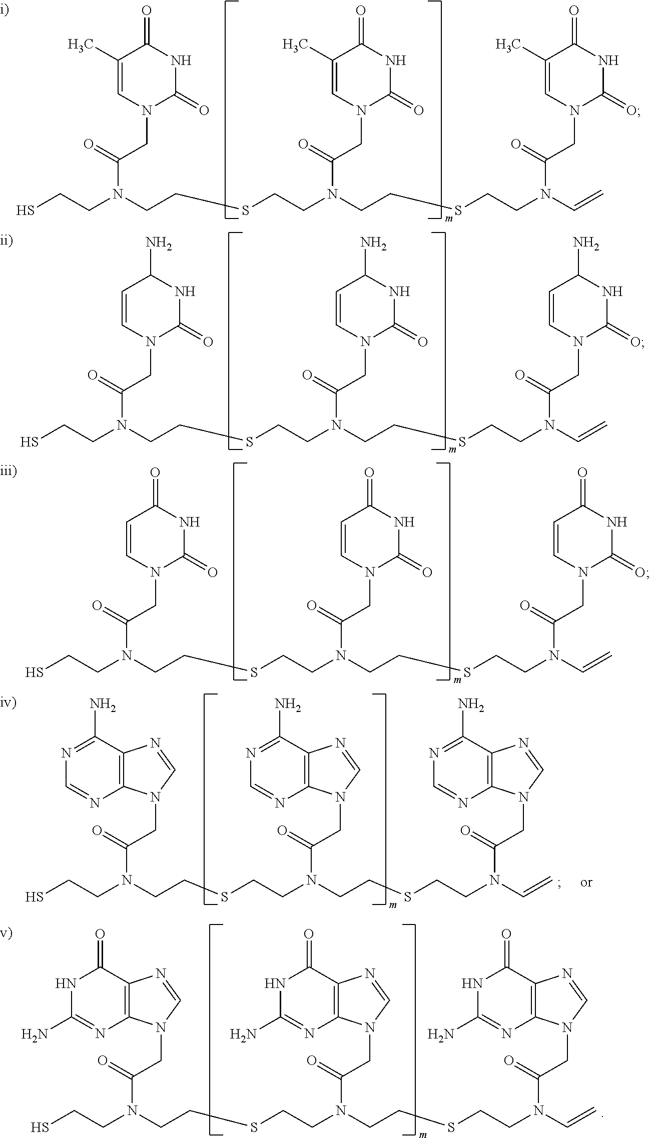

In another iteration of this embodiment, the CNA can comprise from 2 to 10 different Z units. For example, the CNA comprising the nucleobases thymine and cytosine having the formula:

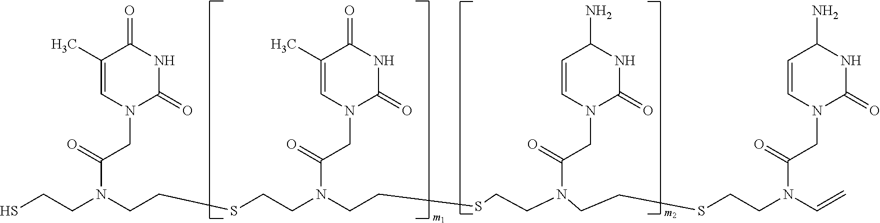

wherein the indices m₁+m₂=m.

In a further aspect, the CNA's can be formed from monomer dimers, for example:

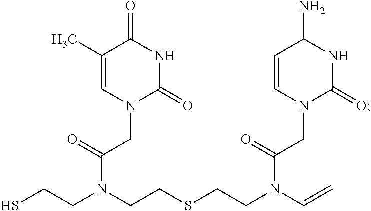 [SMILES: C=CN(CCSCCN(CCS)C(=O)Cn1cc(C)c(=O)[nH]c1=O)C(=O)CN1C=CC(N)NC1=O]

which can provide CNA's having the formula:

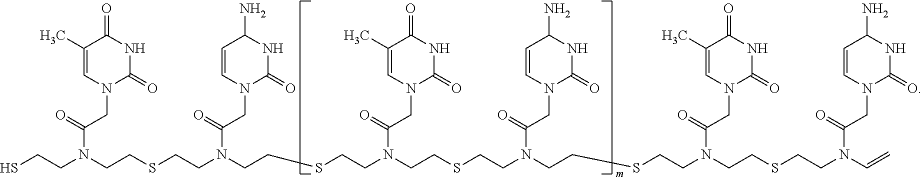

In one non-limiting example, a CNA according this iteration wherein m is equal to 1, has the formula:

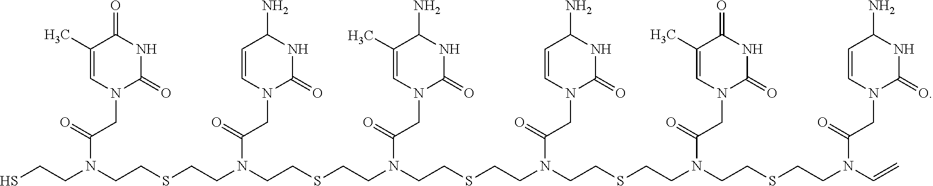 [SMILES: C=CN(CCSCCN(CCSCCN(CCSCCN(CCSCCN(CCSCCN(CCS)C(=O)Cn1cc(C)c(=O)[nH]c1=O)C(=O)CN1C=CC(N)NC1=O)C(=O)CN1C=C(C)C(N)NC1=O)C(=O)CN1C=CC(N)NC1=O)C(=O)Cn1cc(C)c(=O)[nH]c1=O)C(=O)CN1C=CC(N)NC1=O]

In another aspect the disclosed polymers have the formula:

[X₆]—[X₅]n-[X₄]—[X₂]ₘ—[X₃]

wherein X₂ has the formula:

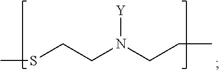

X₃ has the formula:

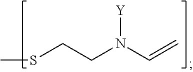

X₄ has the formula:

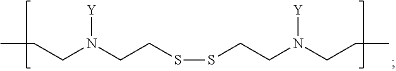

X₅ has the formula:

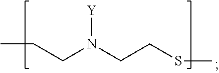

and
X₆ has the formula:

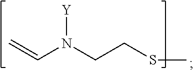

Y has the formula:

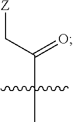

each Z is the same or a different moiety capable of annealing to a complementary moiety; and the indices m and n are integers from 0 to 20. In one embodiment m=n. In another embodiment m>n. In a further embodiment m<n. The indices m and n can have any value from 0 to 20, for example, 0, 1, 2, 3, 4, 5, 6, 7, 8, 9, 10, 11, 12, 13, 14, 15, 16, 17, 18, 19, and 20.

For this aspect the monomers which are used for the previous aspect, i.e., X₂ and X₃ are the same as X₅ and X₆ respectively. Their orientations in the backbone are, however, reversed since the CNA's grow outward from X₄ units.

In one embodiment of this aspect Z is a nucleobase. Non-limiting examples of suitable nucleobases includes cytosine, guanine, adenine, thymine, uracil, 5-fluorouracil, 5-bromouracil, 5-chlorouracil, 5-iodouracil, hypoxanthine, xanthine, acetylcytosine, 5-(carboxyhydroxyl-methyl)uracil, 5-carboxymethylaminomethyl-2-thiouridine, 5-carboxymethylamino-methyluracil, dihydrouracil, β-D-galactosyl-queosine, inosine, 1-methylguanine, 1-methyl-inosine, 2,2-dimethylguanine, 2-methyladenine, 2-methylguanine, 3-methyl-cytosine, 5-methyl-cytosine, N⁶-adenine, 7-methylguanine, 5-methylaminomethyluracil, methoxyamino-methyl-2-thiouracil, beta-D-mannosylqueosine, 5′-methoxycarboxy-methyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine.

The dimers which comprise the X₄ units have the general formula:

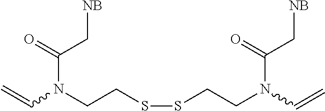

Wherein NB is a nucleobase as defined herein. A non-limiting example of monomers which form the X₄ units of this aspect can be prepared according to Scheme II and Example 2.

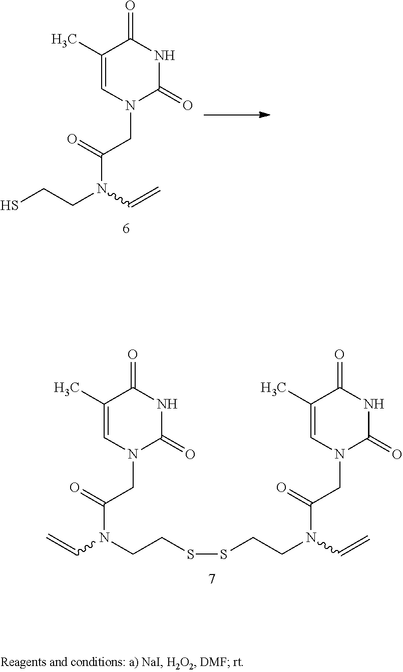 [SMILES: C=CN(CCS)C(=O)Cn1cc(C)c(=O)[nH]c1=O ; C=CN(CCSSCCN(C=C)C(=O)Cn1cc(C)c(=O)[nH]c1=O)C(=O)Cn1cc(C)c(=O)[nH]c1=O]

### Example 2

### Preparation of N,N′-(disulfanediylbis(ethane-2,1-diyl))bis(2-(5-methyl-2,4-dioxo-3,4-dihydropyrimidin-1(2H)-yl)-N-vinylacetamide), 7

To a solution of compound 6 (27.1 mg, 0.1 mmol) in DMF (0.2 mL) were added NaI (15 μL, 10 mg/mL, 0.001 mmol) and H₂O₂(11 μL, 30 wt % in water, 0.1 mmol) successively. The resulting mixture was stirred at room temperature for 30 minutes, then diluted with CH₂Cl₂ (10 mL) and washed with water. The organic layer was separated, dried over MgSO₄, filtered, charged with silica gel, and concentrated under vacuum. The resulting dry silica gel was loaded onto a cartridge and purified by flash chromatography (CH₂Cl₂:MeOH=100:0 to 5:1) to afford dimer (11.2 mg, mixture of diastereomers, 42% yield) as a white solid. ¹H NMR (400 MHz, DMSO-d₆): δ 11.36 (s, 2H), 7.42 (s, 0.71H), 7.40 (1.29H), 7.27-7.14 (m, 0.8H), 6.99-6.91 (m, 1.2H), 4.80-4.73 (m, 6H), 4.52-4.47 (m, 2H), 3.92-3.83 (m, 4H), 3.08-3.00 (m, 1.3H), 2.90-2.81 (m, 2.7H), 1.78-1.74 (m, 6H); HRMS (ESI) m/z calculated for C₂₂H₂₈N₆O₆S₂ [M−H]⁻ 535.1434, found 535.1442.

In one iteration of this embodiment, each Z is independently chosen from adenine, guanine, thymine, uracil and cytosine. The following are non-limiting example of CNA's wherein each Z is the same nucleobase:

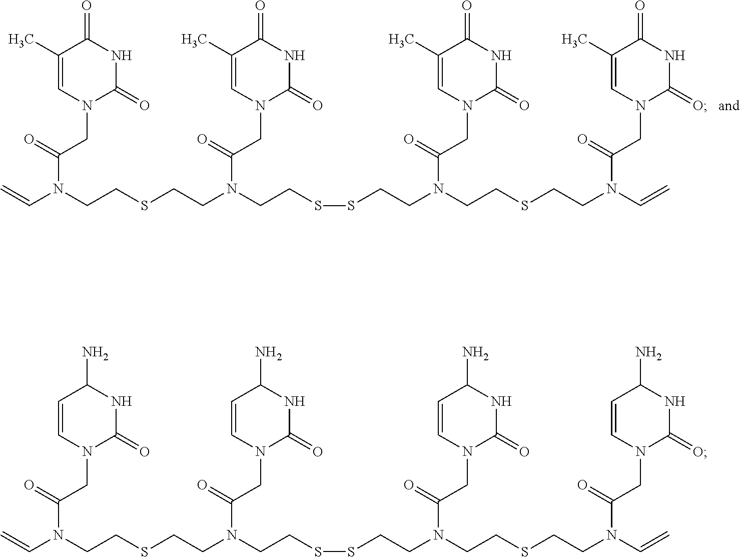 [SMILES: C=CN(CCSCCN(CCSSCCN(CCSCCN(C=C)C(=O)Cn1cc(C)c(=O)[nH]c1=O)C(=O)Cn1cc(C)c(=O)[nH]c1=O)C(=O)Cn1cc(C)c(=O)[nH]c1=O)C(=O)Cn1cc(C)c(=O)[nH]c1=O ; "and" ; C=CN(CCSCCN(CCSSCCN(CCSCCN(C=C)C(=O)CN1C=CC(N)NC1=O)C(=O)CN1C=CC(N)NC1=O)C(=O)CN1C=CC(N)NC1=O)C(=O)CN1C=CC(N)NC1=O]

wherein the indices m and n are both 0.

The disclosed CNA polymers can be formed by the process described in Scheme III and described in Example 3.

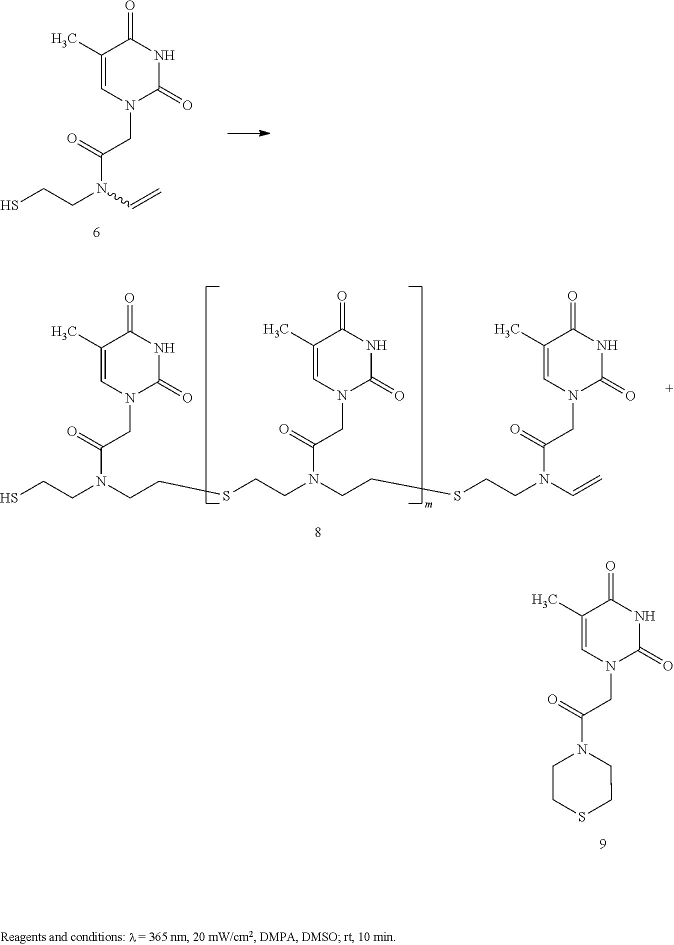

### Example 3

### PolyT Click Nucleic

Preparation of polythymine click nucleic acid polymer, 8: To a mixture of monomer 6 (246.5 mg, 0.92 mmol) and DMSO (920 mg) in 3 mL a glass vial was added a stock solution of 2,2-dimethoxy-2-phenylacetophenone (DMPA) (80 mg, 2.85 wt % in DMSO, 0.0092 mmol). The vial was swirled gently until the mixture formed a homogeneous solution. Then the solution was exposed under LED 365 nm UV light with an intensity of 20 mW/cm² for 10 minutes. The resulting solution was dropwisely added to acetone (45 mL) in a 50 mL centrifuge tube. The white precipitate was washed with acetone (2×45 mL). The precipitate was dried under vacuum to afford polyT CNA (103.5 mg, 42% yield) as a white powder. The precipitate-washed acetone was collected, concentrated, diluted in water, and extracted with CH₂Cl₂ (3×50 mL). The combined organic layers were charged with silica gel and dried under vacuum. The resulting dry silica gel was loaded onto a cartridge and purified by flash chromatography (CH₂Cl₂:MeOH=100:0 to 10:1) to afford cyclic by-product 8 as a white solid. PolyT 7: ¹H NMR (400 MHz, DMSO-d₆): δ 11.33-11.24 (m, 1H), 7.40-7.32 (m, 1H), 4.74-4.59 (m, 2H), 3.53-3.40 (br, 4H), 2.86-2.71 (br, 2H), 2.68-2.60 (br, 2H), 1.73-1.68 (m, 3H). Cyclic by-product 8: ¹H NMR (400 MHz, DMSO-d₆): δ 11.30 (s, 1H), 7.39 (s, 1H), 4.56 (s, 2H), 3.70 (t, 2H, J=4.0 Hz), 2.68 (t, 2H, J=4.0 Hz), 2.55 (t, 2H, J=4.0 Hz), 1.75 (s, 3H); ¹³C NMR (100 MHz, DMSO-d₆): δ 165.4, 164.5, 151.1, 142.4, 108.0, 48.3, 47.0, 44.3, 26.8, 26.4, 12.0; HRMS (ESI) m/z calculated for C₁₁H₁₅N₃O₃S [M+H]⁺ 270.0912, found 270.0917.

Table I discloses the details of several photopolymerizations of compound 6 under varying conditions. Compound 6 (0.2 mmol) was dissolved in DMSO-d₆, followed by adding the calculated amount of DMPA (2.85 wt % in DMSO-d₆). The resulting solution was irradiated with X=365 nm LED light for 10 minutes. The conversion percentage is based on ¹H NMR results. The ratio of compound 7 to compound 8 is based on the integration of characteristic peaks in the vinyl protons as seen in the ¹H NMR spectrum shown in FIG. 2. The average molecular weight Mₙ was determined by size exclusion chromatography.

TABLE I

| | DMPA | Conc. | Conversion | 7/8 | | | % |
|---|---|---|---|---|---|---|---|
| No. | (mol %) | (mol/L) | (%) | ratio | Mₙ (×10³) | PDI | yield |
| 1 | 5 | 0.25 | >99 | 1/1.8 | 4.5 | 1.23 | 36 |
| 2 | 5 | 0.5 | >99 | 1/1.6 | 4.4 | 1.23 | 39 |
| 3 | 5 | 1.0 | >99 | 1/1.2 | 4.1 | 1.25 | 46 |
| 4 | 10 | 1.0 | >99 | 1/1.2 | 4.1 | 1.24 | 46 |
| 5 | 1 | 1.0 | >99 | 1/1.2 | 4.0 | 1.23 | 46 |
| 6 | 0 | 1.0 | 0 | N/A | N/A | N/A | 0 |

Self-cyclized by-product 8 was also observed in 54% yield (by ¹H NMR). This product is due to the different cyclization tendencies of the two diastereomers of monomer 6 as shown below.

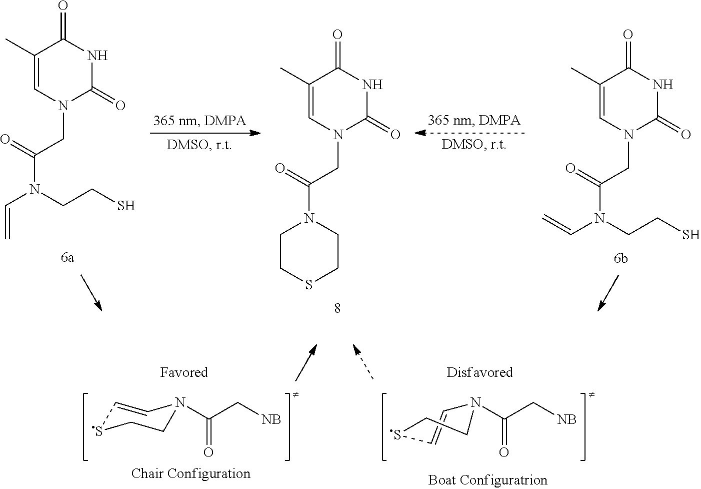

Diastereomer 6a goes through a thermodynamically favored chair-like transition configuration to afford 8, while the disfavored boat-like transition configuration effectively limits the cyclization of 6b. Therefore, cyclization is favored under lower monomer concentration because of the reduced possibilities for cyclization-favored 6a to be captured by the propagating chain. Moreover, the loading of photoinitiator does not influence the Mₙ, PDI, and the polymerization/cyclization ratio significantly (entries 3-5). Each condition of CNA-2G polymerization resulted in polyT 7 with relatively narrow PDI values, which is not feasible from a classic step-growth reaction mechanism but hinted at a more chain-growth-like mechanism. An intramolecular chain-transfer reaction might be involved in both chain growth propagation and cyclization processes. Besides, a control group was arranged to verify the photo-initiated mechanism of this polymerization (entry 6). Without photoinitiator, there was no conversion observed by ¹H NMR after 10 min irradiation, which indicated that the photoinitiator is necessary for this polymerization, at least on these relatively short timescales, though thiol-ene polymerization has been shown to be effective even without photoinitiators under some conditions.

By-product 8 was separated by precipitating the crude solution into acetone, which provided the pure polyT as a white powder in 42% isolated yield. The structure of polyT was confirmed by ¹H NMR spectroscopy as depicted in FIG. 2. With sufficient amplification of the 4.5 to 7.5 ppm region, the characteristic peaks of the terminal vinyl group were observed, consistent with the linear structure of polyT CNA.

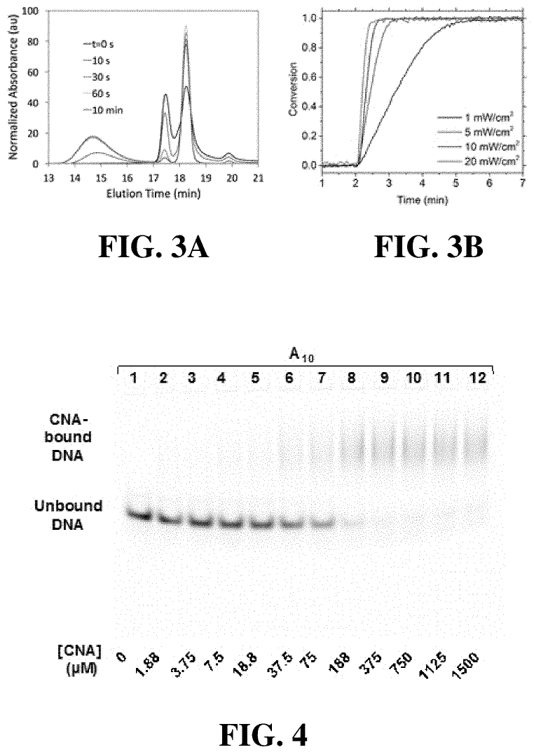

The photopolymerization efficiency determined by size exclusion chromatograpy (SEC) and Fourier Transform Infrared (FTIR) spectroscopy as depicted in FIG. 3A and FIG. 3B. The molecular weight trend evidenced by the SEC for irradiation times of 0-600 s indicated that the polymerization was nearly complete in 30 seconds at room temperature, under 20 mW/cm² intensity of 365 nm light, and with 1 mol % loading of photo-initiator. Relatively high-molecular-weight polymers (Mₙ=3600) were formed within the first ten seconds of light exposure. Increased exposure increased the average molecular weight of the polymers, but did not indicate a greater conversion percentage. These data indicate a chain-growth-like propagation mechanism since the step-growth mechanism exhibited a slow initial evolution of molecular weight which increased at higher conversions. The low molecular-weight peak at 18.2 minutes, sharing an elution time with pure monomer, represents the cyclized byproduct compound 8. The disappearing peak at 17.4 minutes belongs to the disulfide-linked dimer compound 7. Furthermore, a “conversion versus time” relationship was obtained from FTIR based on monitoring the amount of remaining thiol-associated peak at 2540 cm⁻¹ with increasing irradiation time. Consistent with the SEC results, with 1 mol % loading of DMPA and 20 mW/cm² light intensity, nearly quantitative thiol group consumption was achieved in less than 30 seconds. Lowering the light intensity to 1 mW/cm², the polymerization was still completed in 4 minutes.

### Nanoparticle/Click Nucleic Acid Polymers Conjugates

Disclosed herein are click nucleic acids that are conjugates with nanoparticles. One aspect of the disclosed conjugates has the formula:

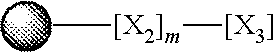

wherein the symbol:
[symbol] represents a nanoparticle, X₂, X₃ and m are the same as defined herein above. The polymers which comprise the conjugates are the same polymers disclosed herein.

The following are non-limiting examples of the disclosed nanoparticle/click nucleic acid polymer conjugates according to this aspect:

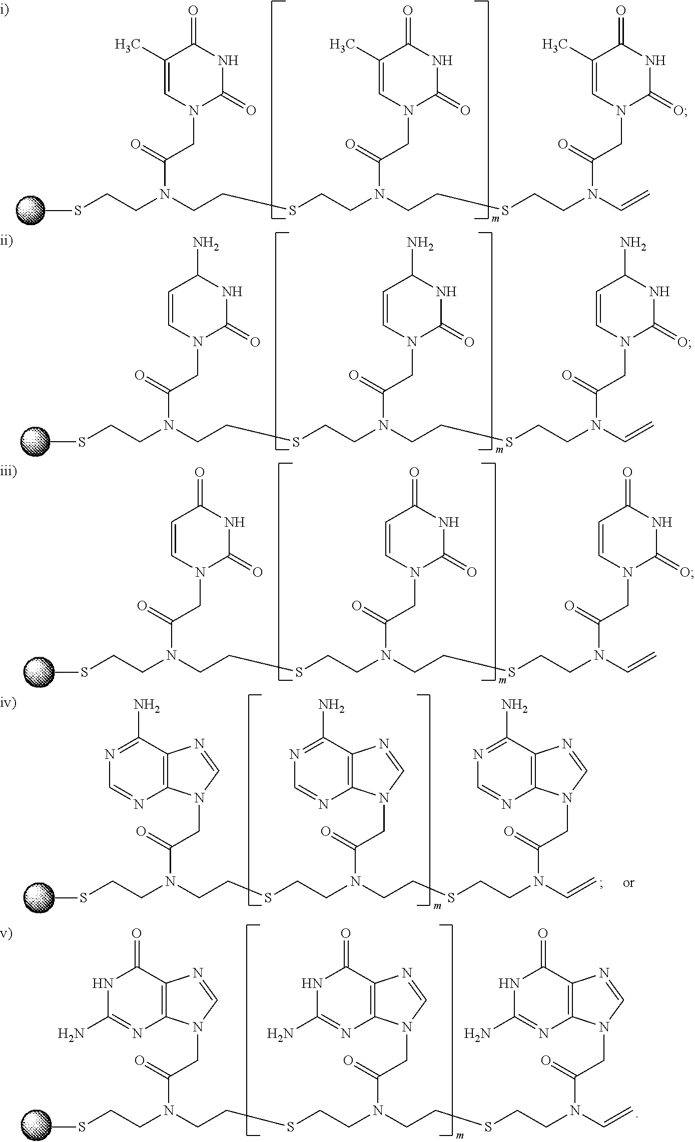

A further aspect of the disclosed conjugates has the formula:

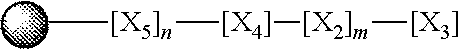

wherein X₂, X₃, X₄, X₅, and the indices m and n are the same as defined herein above. The following are non-limiting examples of the disclosed nanoparticle/click nucleic acid polymer conjugates according to this aspect:
i)

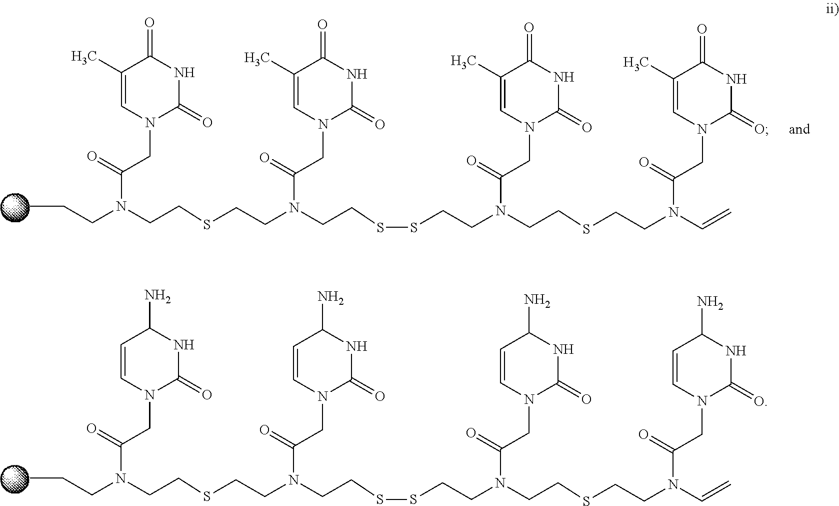 [SMILES: C=CN(CCSCCN(CCSSCCN(CCSCCN(CCC)C(=O)Cn1cc(C)c(=O)[nH]c1=O)C(=O)Cn1cc(C)c(=O)[nH]c1=O)C(=O)Cn1cc(C)c(=O)[nH]c1=O)C(=O)Cn1cc(C)c(=O)[nH]c1=O ; C=CN(CCSCCN(CCSSCCN(CCSCCN(CCC)C(=O)CN1C=CC(N)NC1=O)C(=O)CN1C=CC(N)NC1=O)C(=O)CN1C=CC(N)NC1=O)C(=O)CN1C=CC(N)NC1=O]

The disclosed conjugates can be prepared according to the procedure described in Example 4.

### Example 4

### Preparation of 10 nm CdS Nanoparticles:

To a 100 mL round-bottom flask were charged with CdCl₂ (47 mg), octadecene (10 mL), and oleic acid (0.93 mL). The resulting solution was purged with argon for 30 minutes, followed by heating to 120° C. and maintaining at this temperature for 30 minutes. Then, a solution of sulfur (8 mg) in oleic acid (822 μL) was added in one portion and the resulting mixture was heated to 220° C. in 10 minutes. The oleic acid stabilized CdS nanoparticles were collected by centrifugation, then washed with toluene and acetone.

### Preparation of CdS-CNA Nanoparticles:

To a solution of oleic acid stabilized CdS nanoparticles (4.6 mg) in chloroform (3 mL) were added NaOH aqueous solution (2 mL, 50 mM) and mercaptoethanol (50 μL) successively. The resulting mixture was stirred at room temperature overnight, followed by centrifugation in 30K filter. The precipitate was collected and re-dispersed in DMSO/water (1/1, 0.8 mL) to form a mercaptoethanol-modified CdS nanoparticle stock solution with a nanoparticle concentration of 7.69 μM, which was determined by UV/Vis spectra (absorption peak at 472 nm, extinction coefficient=2.56×10⁶ mol/(L·cm)). To the freshly made mercaptoethanol-modified CdS nanoparticle stock solution (7.69 μM, 30 μL) were added a solution of polyT CNA in DMSO (6.9 μL, 5 mM), a solution of 2K PEG-SH in water (2.3 μL, 5 mM), water (4.6 μL), DMSO (5.5 μL), and NaHCO₃ aqueous solution (5.5 μL, 500 mM) successively. The resulting solution was stirred at room temperature for 24 hours, followed by centrifugation in 30K filter. The precipitate was collected and re-dispersed in DMSO/water (1/1, 25 μL) to form a CdS-CNA nanoparticle stock solution with a concentration of 7.69 μM. Approximately 94 CNA polymers are on each CdS nanoparticle, which was determined by CNA concentration standard curve (Figure S5).

The disclosed CdS-CNA nanoparticles can bind to sections of complementary ssDNA or ssRNA. Without wising to be limited by theory, the ability of the disclosed CdS-CNA nanoparticles to bind with high specificity to complementary ssDNA is a vital characteristic of the disclosed conjugates to serve as DNA mimics, which is requisite for multiple applications. The interaction between the disclosed CdS polyT conjugate and and example of ssDNA was evaluated by gel electrophoresis.

### CdS-CNA Nanoparticle Self-Assembly:

To a CdS polyT conjugate nanoparticle stock solution (3.9 μL, 7.69 μM) were added a DNA-A5 linker aqueous solution (3.0 μL, 463.3 μM), DNA-complementary linker aqueous solution (3.1 μL, 454.0 μM), DMSO (8.1 μL), and NaCl aqueous solution (2.0 μL, 5 M) successively. The resulting solution was annealed at 60° C. for 10 min. After annealing, the solution was cooled down to room temperature at a rate of 1° C./10 min, then cooled down to 4° C. in 1 hour. The resulting solution was kept at 4° C. for further characterization by TEM imagining.

### Polymer Binding

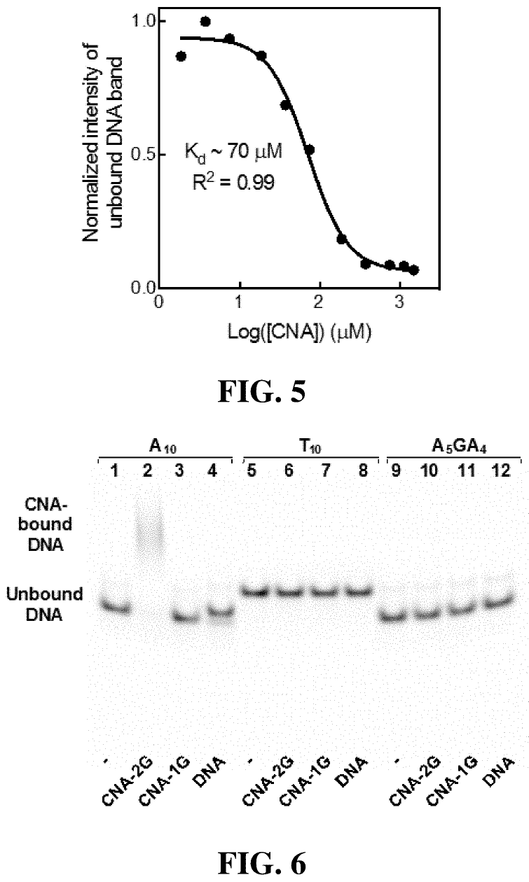

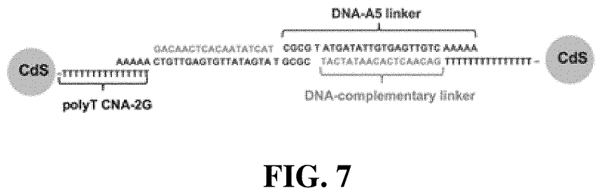

The interaction between polyT polymers and ssDNA was evaluated by gel electrophoresis (FIG. 4). PolyT CNA polymers and polyadenine (polyA) ssDNA were mixed in various quantitative ratios (0 to 300 equivalents of polyT CNA relative to polyA ssDNA) in 50% DMSO and 50% 0.5× Tris/Borate/EDTA (TBE) buffer. After annealing, mixtures were loaded onto 20% Novex TBE polyacrylamide gels (FIG. 4). The results indicated a clear hybridization at ratios of polyT CNA/polyA ssDNA greater than 7.5 (FIG. 4, Lane 6). Increasing the relative excess of the CNA led to a more completely hybridized fraction of the polyA ssDNA (FIG. 4, Lanes 7-12). The intensity of the unbound DNA band in each lane was quantified by using the gel analysis feature in ImageJ software based on the fluorescence intensity of bands and plotted against the polyT CNA concentration. The data were fit to a sigmoidal curve and the K_{d} of polyT CNA-2G/polyA ssDNA interaction was determined under these conditions to be approximately 70 μM (FIG. 5).

The specificity of the disclosed polyT CNA polymers to anneal with DNA was determined by mixing polyT CNA with complementary and non-complementary ssDNA (FIG. 6). PolyT CNA polymers bound to polyA ssDNA (FIG. 6, Lane 2), but no binding was observed for a complete mismatch ssDNA (T₁₀) (FIG. 6, Lane 5) or even a single base-pair mismatch ssDNA sequence (A₅GA₄) (FIG. 6, Lane 9). These results confirmed the binding between polyT CNA polymers and complementary ssDNA was sequence specific. For polyT CNA polymers, on the other hand, no binding was observed even for the complementary ssDNA (FIG. 6, Lanes 3, 7, and 11). PolyT ssDNA was included as a positive control and also showed sequence specific binding (FIG. 6, Lanes 4, 8, and 12).

### Methods

Disclosed herein is a method for preparing a monomer having the formula:

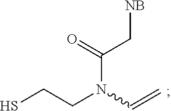

suitable for use in preparing Click Nucleic Acid (CNA) polymers, comprising:
A) reacting 2-chloro-N-(2-chloroethyl)-N-vinylacetamide with a nucleobase to form a compound having the formula:

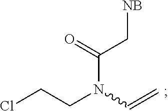

wherein NB is a nucleobase;
B) reacting the compound formed in step (A) with potassium thioacetate under Finkelstein reaction conditions to form a compound having the formula:

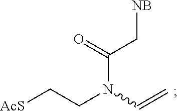

and
C) reacting the compound formed in step (B) with a base to form a monomer having the formula:

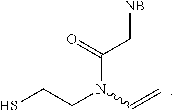

In one aspect the nucleobase in step (A) is chosen from cytosine, guanine, adenine, thymine, uracil, 5-fluorouracil, 5-bromouracil, 5-chlorouracil, 5-iodouracil, hypoxanthine, xanthine, acetylcytosine, 5-(carboxy-hydroxylmethyl)uracil, 5-carboxymethylaminomethyl-2-thiouridine, 5-carboxymethyl-aminomethyluracil, dihydrouracil, β-D-galactosyl-queosine, inosine, 1-methylguanine, 1-methylinosine, 2,2-dimethylguanine, 2-methyladenine, 2-methylguanine, 3-methyl-cytosine, 5-methyl-cytosine, N⁶-adenine, 7-methylguanine, 5-methylaminomethyl-uracil, methoxyamino-methyl-2-thiouracil, beta-D-mannosylqueosine, 5′-methoxycarboxy-methyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine.

In one embodiment of this aspect the nucleobase is chosen from adenine, guanine, thymine, uracil and cytosine.

What is meant by the term “Finkelstein Conditions” is a reaction wherein a more labile leaving group replaces the chlorine atom to facilitate the addition of the thiolacetate moiety. Non-limiting examples of compounds which are useful under Finkelstein Conditions includes NaI, NaBr, KI, KBr, p-toluene sulfonyl chloride (tosyl chloride), methanesulfonyl chloride (mesyl chloride) and the like. In one non-limiting example NaI is used in step (B) and the reaction is conducted in the presence of an organic base and a solvent. Suitable organic bases includes 1,8-diazabicyclo(5.4.0)undec-7-ene (DBU), trimethylamine, 4-dimethylamino-pyridine, and the like.

In one non-limiting example, step (B) recites:
B) reacting the compound formed in step (A) with potassium thioacetate in the presence of DBU and dichloromethane to form a compound having the formula:

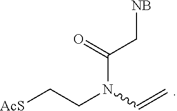

As it relates to step (C), any base capable of hydrolyzing the thiolacetate unit to a thiol is suitable for use. Non-limiting examples include NaOH, KOH and the like.

In one non-limiting example step (C) recites:
C) reacting the compound formed in step (B) with NaOH in the presence of methanol to form a monomer having the formula:

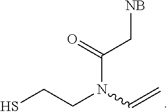

Further disclosed herein is a method for preparing a monomer having the formula:

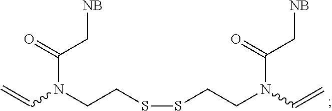

wherein NB is a nucleobase as disclosed herein, said monomer suitable for use in preparing Click Nucleic Acid (CNA) polymers. The process comprising, reacting a monomer having the formula:

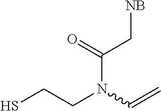

with NaI and hydrogen peroxide in a solvent to form a compound having the formula:

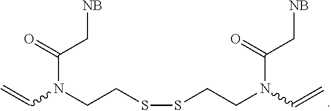

Further disclosed herein are methods for preparing the disclosed polymers. One aspect relates to the preparation of polymers having the formula:

[X₁]—[X₂]ₘ—[X₃].

The method comprises:
A) irradiating a monomer having the formula:

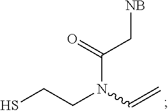

with a source of radiation to form a reaction product admixture comprising:
i) a click nucleic acid polymer having the formula:

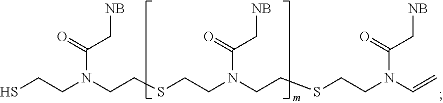

and
ii) a thiomorpholine by-product having the formula:

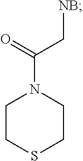

and
B) adding the admixture to a solvent which precipitates the desired oligomer; wherein the index m is an integer from 0 to 20.

In one embodiment, the thiomorpholine by product is collected by filtration and the click nucleic acid polymer is then isolated.

In one embodiment, the monomer in step (A) is irradiated with ultra violet radiation in the presence of a photo initiator. In one non-limiting example, the monomer is irradiated with radiation having X=365 nm for 10 minutes in the presence of 2,2-dimethoxy-2-phenylaceto-phenone.

The reaction variables, i.e., amount of any photo initiator, reaction time, concentration of monomer, time, choice of solvent can be varied by the formulator depending upon the monomer type and degree of polymerization desired or the amount of thiomorpholine by-product.

Further disclosed are methods for preparing the disclosed nanoparticle click nucleic acid polymer conjugate, having the formula:

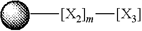

wherein the symbol:
[symbol] represents a nanoparticle.

The disclosed process comprises:
A) reacting a nanoparticle precursor with one or more surface modifying agents to form a surface modified nanoparticle;
B) reacting the surface modified nanoparticle with mercaptoethanol to form a mercaptoethanol-modified nanoparticle; and
C) reacting the mercaptoethanol-modified nanoparticle with a CNA polymer to form a nanoparticle click nucleic acid polymer conjugate.

In one embodiment, step (A) recites:
A) reacting CdCl₂ with octadecene, oleic acid and sulfur to form an oleic acid stabilized CdS nanoparticle.

In one iteration step (A) is conducted at a temperature of from about 100° C. to about 250° C. In one non-limiting example, CdCl₂, octadecene, and oleic acid are reacted together at a temperature of from about 100° C. to about 150° C. after which sulfur is added and the temperature is increased to from about 200° C. to about 250° C.

In one iteration of this embodiment step (B) recites:
B) reacting oleic acid stabilized CdS nanoparticle with mercaptoethanol, an inorganic base in a solvent to form a mercaptoethanol-modified CdS nanoparticle.

In one iteration of this embodiment step (C) recites:
C) reacting the mercaptoethanol-modified CdS nanoparticle with a CNA polymer chosen from polyT, polyA, polyC, polyG or polyU in the presence of solvent and an inorganic base to form a nanoparticle click nucleic acid polymer conjugate.

In one non-limiting example step (C) comprises conducting the reaction in a water soluble organic solvent and water in the presence of a polyethylene-SH surfactant and a weak inorganic base. For example, the mercaptoethanol-modified CdS nanoparticles are added a solution of polyT CNA in DMSO, wherein an aqueous solution of 2K PEG-SH, water (4.6 μL), DMSO, and aqueous NaHCO₃ are added successively.

The disclosed nanoparticle click nucleic acid polymer conjugates according to this embodiment include conjugates having the formula:

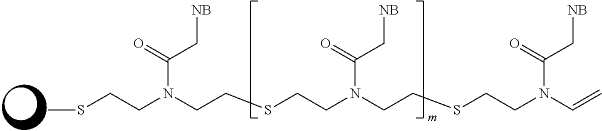

wherein NB is a nucleobase chosen from adenine, guanine, thymine, uracil or cytosine and the nanoparticle is CdS.

What is claimed is:

1. A nanoparticle click nucleic acid polymer conjugate, having the formula: 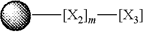
wherein the symbol: [symbol]
represents a nanoparticle;
X₂ has the formula: 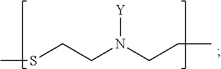
X₃ has the formula: 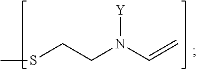
Y has the formula: 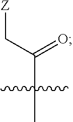
each Z is the same or a different moiety capable of annealing to a complementary moiety; and
the index m is an integer from 0 to 20.

2. The conjugate according to claim 1, wherein Z is a nucleobase.

3. The conjugate according to claim 1, wherein the nucleobase is chosen from cytosine, guanine, adenine, thymine, uracil, 5-fluorouracil, 5-bromouracil, 5-chlorouracil, 5-iodouracil, hypoxanthine, xanthine, acetylcytosine, 5-(carboxy-hydroxylmethyl)uracil, 5-carboxymethylaminomethyl-2-thiouridine, 5-carboxymethyl-aminomethyluracil, dihydrouracil, β-D-galactosyl-queosine, inosine, 1-methylguanine, 1-methylinosine, 2,2-dimethylguanine, 2-methyladenine, 2-methylguanine, 3-methyl-cytosine, 5-methyl-cytosine, N⁶-adenine, 7-methylguanine, 5-methylaminomethyluracil, methoxyamino-methyl-2-thiouracil, beta-D-mannosylqueosine, 5′-methoxycarboxy-methyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine

4. The conjugate according claim 1, wherein each Z is independently chosen from adenine, guanine, thymine, uracil and cytosine.

5. A conjugate according claim 1, having the formula chosen from:

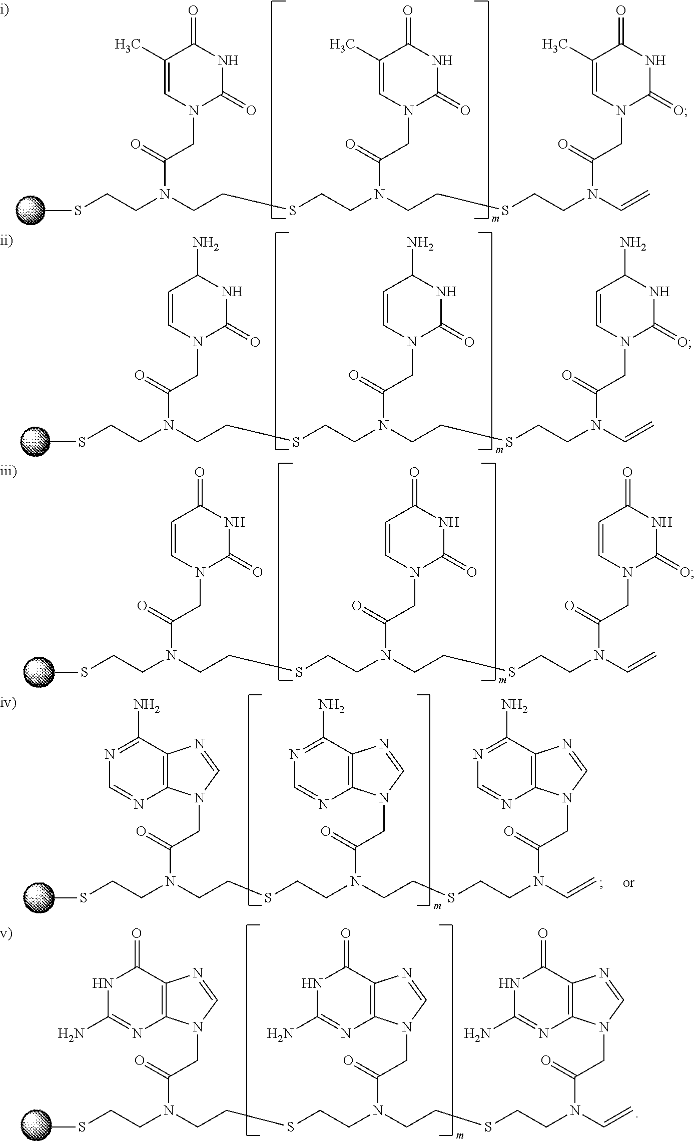

6. The conjugate according claim 1, wherein the nanoparticle is chosen from gold, CdS, CdSe, CdSe/ZnS, CIS, InP/ZnS, CdTeSeS or SiO₂.

7. The conjugate according claim 6, wherein the nanoparticle is gold.

8. The conjugate according claim 6, wherein the nanoparticle is CdS.

9. The conjugate according claim 1, wherein the nanoparticle is a surface modified nanoparticle.

10. A nanoparticle click nucleic acid polymer conjugate, having the formula: 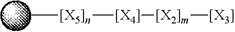
wherein the symbol: [symbol]
represents a nanoparticle;
X₂ has the formula: 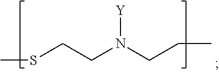
X₃ has the formula: 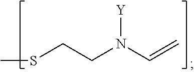
X₄ has the formula: 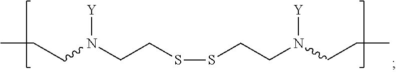
X₄ has the formula: 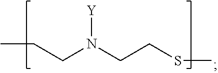
and
Y has the formula: 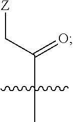
each Z is the same or a different moiety capable of annealing to a complementary moiety; and
the indices m and n are integers independently from 0 to 20.

11. The conjugate according to claim 10, wherein Z is a nucleobase.

12. The conjugate according to claim 10, wherein the nucleobase is chosen from cytosine, guanine, adenine, thymine, uracil, 5-fluorouracil, 5-bromouracil, 5-chlorouracil, 5-iodouracil, hypoxanthine, xanthine, acetylcytosine, 5-(carboxy-hydroxylmethyl)uracil, 5-carboxymethylaminomethyl-2-thiouridine, 5-carboxymethyl-aminomethyluracil, dihydrouracil, β-D-galactosyl-queosine, inosine, 1-methylguanine, 1-methylinosine, 2,2-dimethylguanine, 2-methyladenine, 2-methylguanine, 3-methyl-cytosine, 5-methyl-cytosine, N⁶-adenine, 7-methylguanine, 5-methylaminomethyluracil, methoxyamino-methyl-2-thiouracil, beta-D-mannosylqueosine, 5′-methoxycarboxy-methyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine,

13. The conjugate according claim 10, wherein each Z is independently chosen from adenine, guanine, thymine, uracil and cytosine.

14. A conjugate according claim 10, having the formula chosen from:

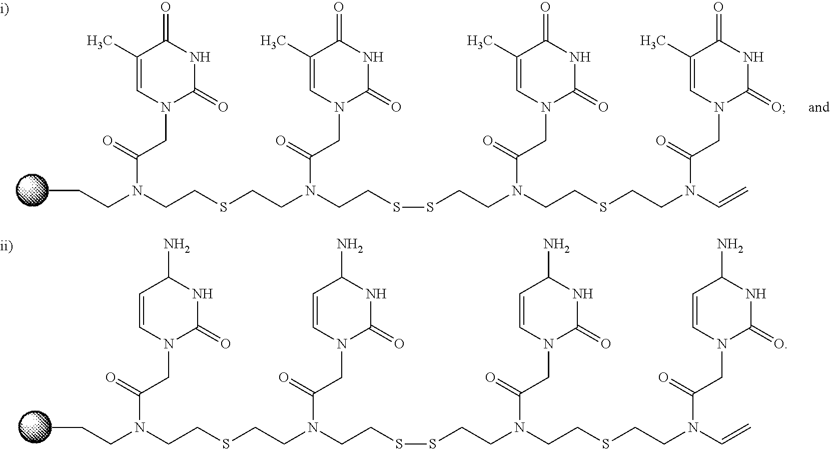

15. The conjugate according claim 10, wherein the nanoparticle is chosen from gold, CdS, CdSe, CdSe/ZnS, CIS, InP/ZnS, CdTeSeS or SiO₂.

16. The conjugate according claim 10, wherein the nanoparticle is gold.

17. The conjugate according claim 10, wherein the nanoparticle is CdS.

18. The conjugate according claim 10, wherein the nanoparticle is a surface modified nanoparticle.

19. A process for preparing a monomer having the formula: 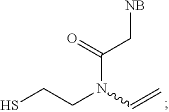
suitable for use in preparing Click Nucleic Acid (CNA) polymers, comprising:
A) reacting 2-chloro-N-(2-chloroethyl)-N-vinylacetamide with a nucleobase to form a compound having the formula: 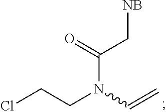
wherein NB is a nucleobase chosen from adenine, guanine, thymine, uracil or cytosine;
B) reacting the compound formed in step (A) with potassium thioacetate under Finkelstein reaction conditions to form a compound having the formula: 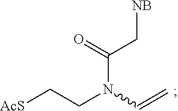
and
C) reacting the compound formed in step (B) with a base to form a monomer having the formula:

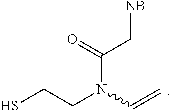

20. The process according to claim 19, wherein step (C) further comprises a reagent chosen from NaI, NaBr, KI, KBr, p-toluene sulfonyl chloride, or methanesulfonyl chloride and an organic base chosen from 1,8-diazabicyclo(5.4.0)undec-7-ene, trimethylamine, or 4-dimethylaminopyridine.

21. The process according claim 19, wherein the nucleobase in Step (A) is chosen form cytosine, guanine, adenine, thymine, uracil, 5-fluorouracil, 5-bromouracil, 5-chlorouracil, 5-iodouracil, hypoxanthine, xanthine, acetylcytosine, 5-(carboxy-hydroxylmethyl)uracil, 5-carboxymethylaminomethyl-2-thiouridine, 5-carboxymethyl-aminomethyluracil, dihydrouracil, β-D-galactosyl-queosine, inosine, 1-methylguanine, 1-methylinosine, 2,2-dimethylguanine, 2-methyladenine, 2-methylguanine, 3-methyl-cytosine, 5-methyl-cytosine, N⁶-adenine, 7-methylguanine, 5-methylaminomethyl-uracil, methoxyamino-methyl-2-thiouracil, beta-D-mannosylqueosine, 5′-methoxy-carboxymethyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine.

22. The process according claim 19, wherein NB is a nucleobase chosen from adenine, guanine, thymine, uracil or cytosine.

23. The process according claim 19, wherein step (B) is conducted in the presence of 1,8-diazabicyclo(5.4.0)undec-7-ene, trimethylamine and dichloromethane.

24. The process according claim 19, wherein the monomer formed is chosen from:
i) N-(2-mercaptoethyl)-2-(5-methyl-2,4-dioxo-3,4-dihydropyrimidin-1(2H)-yl)-N-vinylacetamide having the formula: 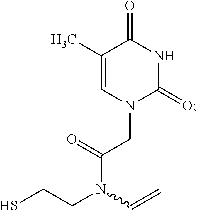 [SMILES: C=CN(CCS)C(=O)Cn1cc(C)c(=O)[nH]c1=O]
ii) 2-(4-amino-2-oxo-3,4-dihydropyrimidin-1 (2H)-yl)-N-(2-mercaptoethyl)-N-vinylacetamide having the formula: 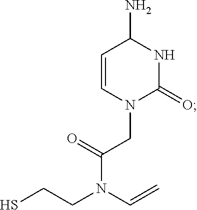 [SMILES: C=CN(CCS)C(=O)CN1C=CC(N)NC1=O]
iii) 2-(2,4-Dioxo-3,4-dihydropyrimidin-1 (2H)-yl)-N-(2-mercaptoethyl)-N-vinylacetamide having the formula: 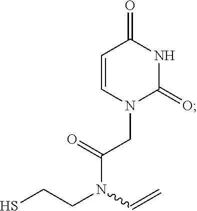 [SMILES: C=CN(CCS)C(=O)Cn1ccc(=O)[nH]c1=O]
iv) 2-(6-Amino-9H-purin-9-yl)-N-(2-mercaptoethyl)-N-vinylacetamide having the formula:
and 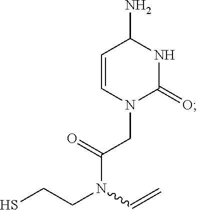 [SMILES: C=CN(CCS)C(=O)CN1C=CC(N)NC1=O]
v) 2-(2-Amino-6-oxo-1H-purin-9(6H)-yl)-N-(2-mercaptoethyl)-N-vinylacetamide having the formula:

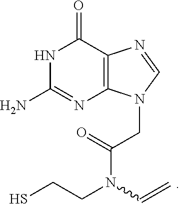 [SMILES: C=CN(CCS)C(=O)Cn1cnc2c(=O)[nH]c(N)nc21]

25. A process for preparing a click nucleic acid polymer having the formula:
[X₁]—[X₂]ₘ—[X₃];
said process comprising:
A) irradiating a monomer having the formula: 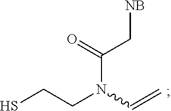
with a source of radiation to form a reaction product admixture comprising:
i) a click nucleic acid polymer having the formula: 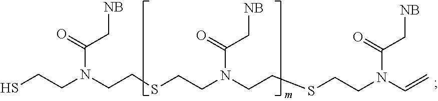
and
ii) a thiomorpholine by-product having the formula: 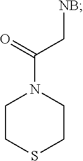
and
B) adding the admixture to a solvent which precipitates the click nucleic acid polymer;
NB is a nucleobase and the index m is an integer from 0 to 20.

26. The process according to claim 25, wherein the nucleobase in Step (A) is chosen form cytosine, guanine, adenine, thymine, uracil, 5-fluorouracil, 5-bromouracil, 5-chlorouracil, 5-iodouracil, hypoxanthine, xanthine, acetylcytosine, 5-(carboxy-hydroxylmethyl)uracil, 5-carboxymethylaminomethyl-2-thiouridine, 5-carboxymethyl-aminomethyluracil, dihydrouracil, (β-D-galactosyl-queosine, inosine, 1-methylguanine, 1-methylinosine, 2,2-dimethylguanine, 2-methyladenine, 2-methylguanine, 3-methyl-cytosine, 5-methyl-cytosine, N⁶-adenine, 7-methylguanine, 5-methylaminomethyl-uracil, methoxyamino-methyl-2-thiouracil, beta-D-mannosylqueosine, 5′-methoxy-carboxymethyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine.

27. The process according claim 25, wherein NB is a nucleobase chosen from adenine, guanine, thymine, uracil or cytosine.

28. The process according claim 25, wherein the solvent in step (B) is acetone.

29. The process according claim 25, wherein the polymer has the formula chosen from:

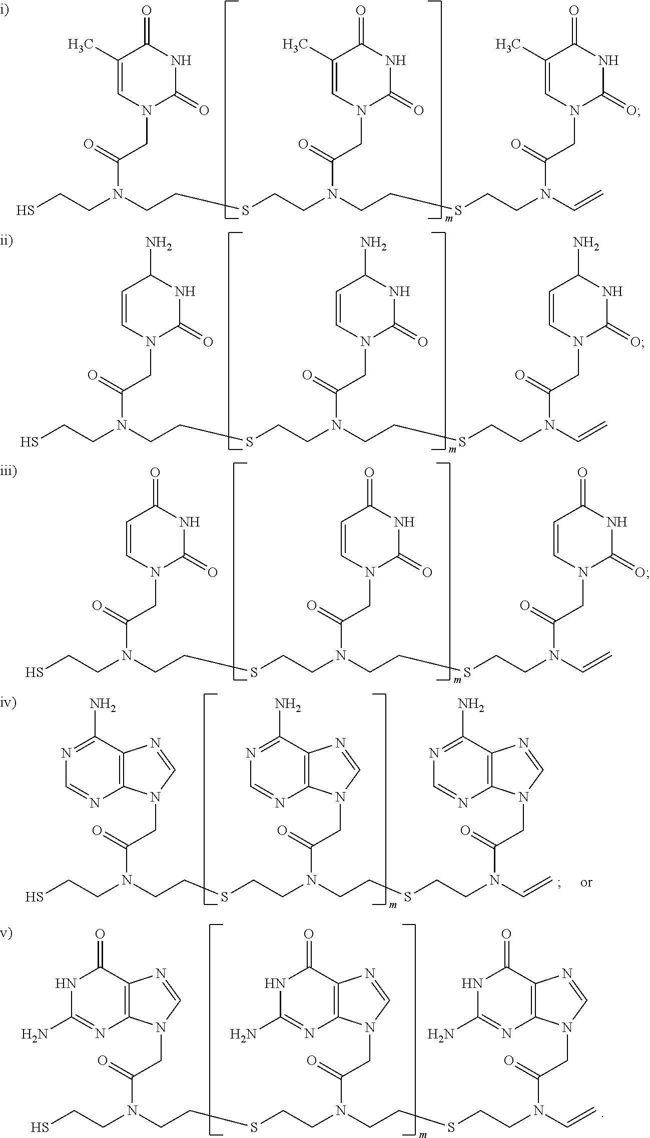

30. The process according claim 25, wherein step (A) further comprises a photo initiator.

31. A process for forming a nanoparticle click nucleic acid conjugate having the formula: 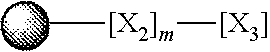
wherein the symbol: [symbol]
represents a nanoparticle;
X₂ has the formula: 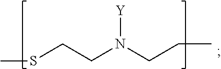
X₃ has the formula: 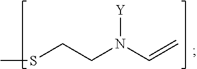
Y has the formula: 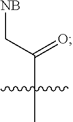
Z is a nucleobase; and
m is an integer from 0 to 20;
wherein said method comprises:
A) reacting a nanoparticle precursor with one or more surface modifying agents to form a surface modified nanoparticle;
B) reacting the surface modified nanoparticle with mercaptoethanol to form a mercaptoethanol-modified nanoparticle; and
C) reacting the mercaptoethanol-modified nanoparticle with a CNA polymer to form a nanoparticle click nucleic acid polymer conjugate.

32. The process according to claim 31, wherein the nucleobase in Step (A) is chosen form cytosine, guanine, adenine, thymine, uracil, 5-fluorouracil, 5-bromouracil, 5-chlorouracil, 5-iodouracil, hypoxanthine, xanthine, acetylcytosine, 5-(carboxy-hydroxylmethyl)uracil, 5-carboxymethylaminomethyl-2-thiouridine, 5-carboxymethyl-aminomethyluracil, dihydrouracil, β-D-galactosyl-queosine, inosine, 1-methylguanine, 1-methylinosine, 2,2-dimethylguanine, 2-methyladenine, 2-methylguanine, 3-methyl-cytosine, 5-methyl-cytosine, N⁶-adenine, 7-methylguanine, 5-methylaminomethyl-uracil, methoxyamino-methyl-2-thiouracil, beta-D-mannosylqueosine, 5′-methoxy-carboxymethyluracil, 5-methoxyuracil, 2-methylthio-N⁶-isopentenyladenine, uracil-5-oxyacetic acid, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N²-carboxypropyl)uracil, or 2,6-diaminopurine.

33. The process according claim 31, wherein NB is a nucleobase chosen from adenine, guanine, thymine, uracil or cytosine.

34. The process according claim 31, wherein step (A) comprises:
A) reacting CdCl₂ with octadecene, oleic acid and sulfur to form an oleic acid stabilized CdS nanoparticle.

35. The process according to claim 34, wherein step (B) comprises:
B) reacting the oleic acid stabilized CdS nanoparticle with mercaptoethanol, and an inorganic base in a solvent to form a mercaptoethanol-modified CdS nanoparticle.

36. The process according to claim 35, wherein step (C) comprises:
C) reacting the mercaptoethanol-modified CdS nanoparticle with a CNA polymer chosen from polyT, polyA, polyC, polyG or polyU in the presence of solvent and an inorganic base to form a nanoparticle click nucleic acid polymer conjugate.

37. The process according claim 31, wherein the conjugate has the formula:

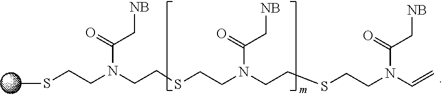

38. A process according claim 31, wherein the conjugate has the formula chosen from:

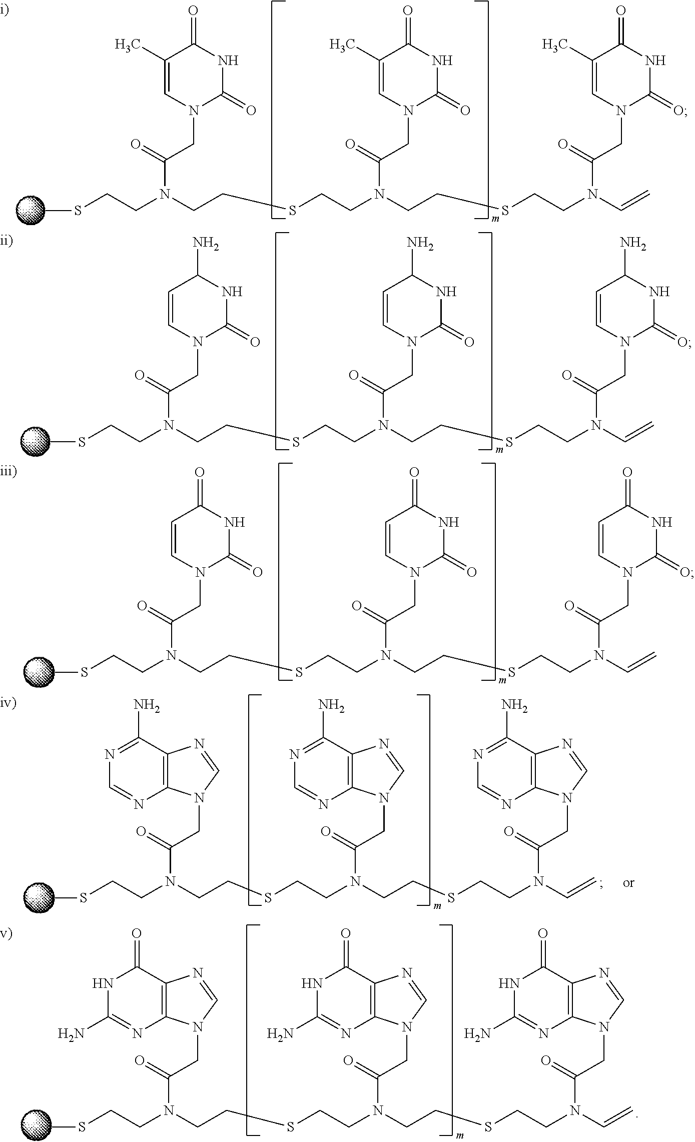

39. The conjugate according claim 31, wherein the nanoparticle is chosen from gold, CdS, CdSe, CdSe/ZnS, CIS, InP/ZnS, CdTeSeS or SiO₂.

40. The conjugate according claim 39, wherein the nanoparticle is gold.

41. The conjugate according claim 39, wherein the nanoparticle is CdS.

42. The conjugate according claim 31, wherein the nanoparticle is a surface modified nanoparticle.
